(19) United States
(12) Patent Application Publication
(10) Pub. No.: US 2009/0186360 A1
(43) Pub. Date: Jul. 23, 2009

(54) Detection of GSTP1 hypermethylation in prostate cancer

(76) Inventors: Abhijit Mazumder, Basking Ridge, NJ (US); Shobha A. Varde, Jacksonville, FL (US); Janet M. Vargo, Bridgewater, NJ (US); Jonathan F. Baden, Bridgewater, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/355,869

(22) Filed: Jan. 19, 2009

Related U.S. Application Data
(60) Provisional application No. 61/022,600, filed on Jan. 22, 2008.

(51) Int. Cl. C12Q 1/68

(52) U.S. Cl. 435/6

(57) ABSTRACT

An assay for detecting prostate cancer includes reagents for detecting multiple methylation markers from within one gene such as GSTP1.

### BACKGROUND OF THE INVENTION

This invention relates to the interrogation of methylated genes in concert with other diagnostic methods and kits for use with these methods.

Epigenetic changes (alterations in gene expression that do not involve alterations in DNA nucleotide sequences) are primarily comprised of modifications in DNA methylation and remodeling of chromatin. Alterations in DNA methylation have been documented in a wide range of tumors and genes. Esteller et al. (2001); Bastian et al. (2004); and Esteller (2005). The extent of methylation at a particular CpG site can vary across patient samples. Jeronimo et al. (2001); and Pao et al (2001).

A number of potential methylation markers have recently been disclosed. Glutathione S-transferases (GSTs) are exemplary proteins in which the methylation status of the genes that express them can have important prognostic and diagnostic value for prostate cancer. The proteins catalyze intracellular detoxification reactions, including the inactivation of electrophilic carcinogens, by conjugating chemically-reactive electrophiles to glutathione. (Pickett et al. (1989); Coles et al. (1990); and Rushmore et al. (1993). Human GSTs, encoded by several different genes at different loci, have been classified into four families referred to as alpha, mu, pi, and theta. Mannervik et al. (1992). Decreased GSTP1 expression resulting from epigenetic changes is often related to prostate and hepatic cancers.

Computational approaches (Das et al. (2006)) and bisulfite sequencing (Chan et al. (2005)) indicate that multiple sites within a CpG island can be methylated and that the extent of methylation can vary across these sites. For example, in oral cancer, differences in the degree of methylation of individual CpG sites were noted for p16, E-cadherin, cyclin A1, and cytoglobin. Shaw et al. (2006). In prostate and bladder tumors, the endothelin receptor B displayed hotspots for methylation. (Pao et al. (2001). In colorectal and gastric cancer, methylation of the edge of the CpG island of the death-associated protein kinase gene was detected in virtually every sample, in contrast to the more central regions. Satoh et al. (2002). The differential distribution of methylation is found the RASSF1A CpG island in breast cancer and methylation may progressively spread from the first exon into the promoter area. Yan et al. (2003); and Strunnikova et al. (2005). RASSF2 has frequent methylation at the 5′ and 3′ edges of the CpG island, with less frequent methylation near the transcription start site. Endoh et al. (2005).

In endometrial carcinoma four GSTP1 designs showed sensitivities between 14% and 24% but the sample sizes were too small to determine if these differences were real. (Chan et al. 2005). Two assay designs increase sensitivity of detection of prostate carcinoma (Nakayama et al. (2003)); however, both designs shared the same reverse primer so there was considerable overlap in the regions interrogated. Differences exist in the percent methylation for different CpG sequences for p16, E-cadherin, cyclin A1, and cytoglobin. Shaw et al. (2006). Differential methylation levels at CpG sites exist in breast cancer. Yan et al. (2003).

An inverse correlation exists between tumor MLH1 RNA expression and MLH1 DNA methylation. Yu et al. (2006). Methylation-positive samples exhibited lower levels of RNA expression of the DAPK gene in lung cancer cell lines. Toyooka et al. (2003). However, those studies examined only one site of methylation so correlations with RNA expression at multiple locations in a CpG island could not be determined. The core region surrounding the transcription start site is an informative surrogate for promoter methylation. Eckhardt et al. (2006).

In squamous cell carcinoma of the esophagus, methylation at individual genes increased in frequency from normal to invasive cancer. (Guo et al. 2006). Methylation of TMS1 (p=0.002), DcR1 (p=−0.01), DcR2 (p=0.03), and CRBP1 (p=0.03) correlate with Gleason score and methylation of CRBP1 correlates with higher stage (p=0.0002) and methylation of Reprimo (p=0.02) and TMS1 (p=0.006) correlated with higher (>8 ng/ml) PSA levels. Suzuki et al. (2006). Methylation status was correlated with the extent of myometrial invasion in endometrial carcinoma. A significantly (p=0.04) higher frequency of ASC methylation in the tumor-adjacent, normal tissue for patients was associated with biochemical recurrence, suggesting a correlation with aggressive disease. Chan et al. (2005). RARb2, PTGS2, and EDNRB may have prognostic value in patients undergoing radical prostatectomy. Bastian et al. (2007).

Methylation-specific PCR (MSP) assays have been performed at multiple sites of two genes known to be methylated in prostate cancer, GSTP1 and RARb2. Lee et al. (1994); Harden et al. (2003); Jeronimo et al. (2004); and Nakayama et al. (2001).

### SUMMARY OF THE INVENTION

In one aspect of the invention, assays based on the CpG island spanning bases 834-1319 of GSTP1 sequence (accession number X08508) are presented. These new designs do not overlap that of the prior art (referred to as Version 1 throughout this specification). New designs are referred to as Version 2 and Version 3 throughout this specification. These assays greatly enhance clinical sensitivity and analytical sensitivity.

### DETAILED DESCRIPTION OF THE INVENTION

Molecular assays that detect the presence of hypermethylation in promoter sequences of several genes that can be indicative of the presence of prostate cancer are known. One such gene is GSTP1 and an assay has been described for example in US Patent Publication 20080254455 incorporated herein in its entirety. The assay focuses on the epigenetic silencing of genes through the methylation of cytosines in CpG islands of promoter due to which gene expression is significantly down-regulated or completely eliminated. The methylation specific PCR (MSP) assay is designed to detect methylated sequences by discriminating between methylated and unmethylated cytosines. Prior to being used in a PCR reaction, genomic DNA is subjected to sodium bisulfite modification which converts all cytosines in unmethylated DNA into Uracil, whereas in methylated DNA only cytosines not preceding guanine get converted into Uracil. All cytosines preceding guanine (in a CpG dinucleotide) remain as cytosine.

Hypermethylation of GSTP1 promoter and its association to prostate cancer has been extensively described in the literature. The assay of the instant invention is a vastly improved assay for detecting methylation in the promoter sequence of GSTP1. The new assay is more sensitive and specific and its use of a combination of more than one amplicon for the same gene boosts reliability. The current invention describes the new designs and their comparison to the existing design with formalin fixed paraffin embedded (FFPE) samples. High sensitivity and high specificity of molecular assays are particularly valuable when working with degraded DNA from FFPE tissues, DNA from the very few prostate cells shed into urine, as well as free-floating DNA in the blood of patients with prostate cancer.

The modification of nucleic acid sequences having the potential to express proteins, peptides, or mRNA (such sequences referred to as “genes”) within the genome has been shown, by itself, to be determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression or modification patterns can provide useful information about the occurrence of important events such as tumorogenesis, metastasis, apoptosis, and other clinically relevant phenomena. Relative indications of the degree to which genes are active or inactive can be found in gene expression or modification profiles.

A sample can be any biological fluid, cell, tissue, organ or portion thereof that contains genomic DNA suitable for methylation detection. A test sample can include or be suspected to include a neoplastic cell, such as a cell from the colon, rectum, breast, ovary, prostate, kidney, lung, blood, brain or other organ or tissue that contains or is suspected to contain a neoplastic cell. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation. A reference sample can be used to establish a reference level and, accordingly, can be derived from the source tissue that meets having the particular phenotypic characteristics to which the test sample is to be compared.

A sample for determining gene modification profiles can be obtained by any method known in the art. Samples can be obtained according to standard techniques from all types of biological sources that are usual sources of genomic DNA including, but not limited to cells or cellular components which contain DNA, cell lines, biopsies, bodily fluids such as blood, sputum, stool, urine, cerebrospinal fluid, ejaculate, tissue embedded in paraffin such as tissue from eyes, intestine, kidney, brain, heart, prostate, lung, breast or liver, histological object slides, and all possible combinations thereof. A suitable biological sample can be sourced and acquired subsequent to the formulation of the diagnostic aim of the marker. A sample can be derived from a population of cells or from a tissue that is predicted to be afflicted with or phenotypic of the condition. The genomic DNA can be derived from a high-quality source such that the sample contains only the tissue type of interest, minimum contamination and minimum DNA fragmentation.

Sample preparation requires the collection of patient samples. Patient samples used in the inventive method are those that are suspected of containing diseased cells such as epithelial cells taken from the primary tumor in a colon sample or from surgical margins. Laser Capture Microdissection (LCM) technology is one way to select the cells to be studied, minimizing variability caused by cell type heterogeneity. Consequently, moderate or small changes in gene expression between normal and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, DNA is extracted and amplified and a cytosine methylation profile is obtained, for genes in the appropriate portfolios.

DNA methylation and methods related thereto are discussed for instance in US patent publication numbers 20020197639, 20030022215, 20030032026, 20030082600, 20030087258, 20030096289, 20030129620, 20030148290, 20030157510, 20030170684, 20030215842, 20030224040, 20030232351, 20040023279, 20040038245, 20040048275, 20040072197, 20040086944, 20040101843, 20040115663, 20040132048, 20040137474, 20040146866, 20040146868, 20040152080, 20040171118, 20040203048, 20040241704, 20040248090, 20040248120, 20040265814, 20050009059, 20050019762, 20050026183, 20050053937, 20050064428, 20050069879, 20050079527, 20050089870, 20050130172, 20050153296, 20050196792, 20050208491, 20050208538, 20050214812, 20050233340, 20050239101, 20050260630, 20050266458, 20050287553 and U.S. Pat. Nos. 5,786,146, 6,214,556, 6,251,594, 6,331,393 and 6,335,165.

DNA modification kits are commercially available, they convert purified genomic DNA with unmethylated cytosines into genomic lacking unmethylated cytosines but with additional uracils. The treatment is a two-step chemical process consisting a deamination reaction facilitated by bisulfite and a desulfonation step facilitated by sodium hydroxide. Typically the deamination reaction is performed as a liquid and is terminated by incubation on ice followed by adding column binding buffer. Following solid phase binding and washing the DNA is eluted and the desulfonation reaction is performed in a liquid. Adding ethanol terminates the reaction and the modified DNA is cleaned up by precipitation. However, both commercially available kits (Zymo and Chemicon) perform the desulfonation reaction while the DNA is bound on the column and washing the column terminates the reaction. The treated DNA is eluted from the column ready for MSP assay.

The step of isolating DNA may be conducted in accordance with standard protocols. The DNA may be isolated from any suitable body sample, such as cells from tissue (fresh or fixed samples), blood (including serum and plasma), semen, urine, lymph or bone marrow. For some types of body samples, particularly fluid samples such as blood, semen, urine and lymph, it may be preferred to firstly subject the sample to a process to enrich the concentration of a certain cell type (e.g. prostate cells). One suitable process for enrichment involves the separation of required cells through the use of cell-specific antibodies coupled to magnetic beads and a magnetic cell separation device.

Prior to the amplifying step, the isolated DNA is preferably treated such that unmethylated cytosines are converted to uracil or another nucleotide capable of forming a base pair with adenine while methylated cytosines are unchanged or are converted to a nucleotide capable of forming a base pair with guanine.

Preferably, following treatment and amplification of the isolated DNA, a test is performed to verify that unmethylated cytosines have been efficiently converted to uracil or another nucleotide capable of forming a base pair with adenine, and that methylated cytosines have remained unchanged or efficiently converted to another nucleotide capable of forming a base pair with guanine.

Preferably, the treatment of the isolated DNA involves reacting the isolated DNA with bisulphite in accordance with standard protocols. In bisulphite treatment, unmethylated cytosines are converted to uracil whereas methylated cytosines will be unchanged. Verification that unmethylated cytosines have been converted to uracil and that methylated cystosines have remained unchanged may be achieved by; (i) restricting an aliquot of the treated and amplified DNA with a suitable restriction enzyme which recognize a restriction site generated by or resistant to the bisulphite treatment, and (ii) assessing the restriction fragment pattern by electrophoresis. Alternatively, verification may be achieved by differential hybridization using specific oligonucleotides targeted to regions of the treated DNA where unmethylated cytosines would have been converted to uracil and methylated cytosines would have remained unchanged. The amplifying step may involve polymerase chain reaction (PCR) amplification, ligase chain reaction amplification and others.

Preferably, the amplifying step is conducted in accordance with standard protocols for PCR amplification, in which case, the reactants will typically be suitable primers, dNTPs and a thermostable DNA polymerase, and the conditions will be cycles of varying temperatures and durations to effect alternating denaturation of strand duplexes, annealing of primers (e.g. under high stringency conditions) and subsequent DNA synthesis.

To achieve selective PCR amplification with bisulphite-treated DNA, primers and conditions may be used to discriminate between a target region including a site or sites of abnormal cytosine methylation and a target region where there is no site or sites of abnormal cytosine methylation. Thus, for amplification only of a target region where the said site or sites at which abnormal cytosine methylation occurs is/are methylated, the primers used to anneal to the bisulphite-treated DNA (i.e. reverse primers) may include a guanine nucleotide at a site at which it will form a base pair with a methylated cytosine. Such primers will form a mismatch if the target region in the isolated DNA has unmethylated cytosine nucleotide (which would have been converted to uracil by the bisulphite treatment) at the site or sites at which abnormal cytosine methylation occurs. The primers used for annealing to the opposite strand (i.e. the forward primers) may include a cytosine nucleotide at any site corresponding to site of methylated cytosine in the bisulphite-treated DNA.

The step of amplifying is used to amplify a target region within the GST-Pi gene and/or its regulatory flanking sequences. The regulatory flanking sequences may be regarded as the flanking sequences 5′ and 3′ of the GST-Pi gene which include the elements that regulate, either alone or in combination with another like element, expression of the GST-Pi gene.

Sites of abnormal cytosine methylation can be detected for the purposes of diagnosing or prognosing a disease or condition by methods which do not involve selective amplification. For instance, oligonucleotide/polynucleotide probes could be designed for use in hybridization studies (e.g. Southern blotting) with bisulphite-treated DNA which, under appropriate conditions of stringency, selectively hybridize only to DNA which includes a site or sites of abnormal methylation of cytosine. Alternatively, an appropriately selected informative restriction enzyme can be used to produce restriction fragment patterns that distinguish between DNA which does and does not include a site or sites of abnormal methylation of cytosine.

The method of the invention can also include contacting a nucleic acid-containing specimen with an agent that modifies unmethylated cytosine; amplifying the CpG containing nucleic acid in the specimen by means of CpG-specific oligonucleotide primers; and detecting the methylated nucleic acid. The preferred modification is the 15 conversion of unmethylated cytosines to another nucleotide that will distinguish the unmethylated from the methylated cytosine. Preferably, the agent modifies unmethylated cytosine to uracil and is sodium bisulfite, however, other agents that modify unmethylated cytosine, but not methylated cytosine can also be used. Sodium bisulfite (NaHSO₃) modification is most preferred and reacts readily with the 5,6-double bond of cytosine, but poorly with methylated cytosine. Cytosine reacts with the bisulfite ion to form a sulfonated cytosine reaction intermediate susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template. Scorpion reporters and reagents and other detection systems similarly distinguish modified from unmodified species treated in this manner.

The primers used in the invention for amplification of a CpG-containing nucleic acid in the specimen, after modification (e.g., with bisulfite), specifically distinguish between untreated DNA, methylated, and non-methylated DNA. In methylation specific PCR (MSPCR), primers or priming sequences for the non-methylated DNA preferably have a T in the 3′ CG pair to distinguish it from the C retained in methylated DNA, and the complement is designed for the antisense primer. MSP primers or priming sequences for non-methylated DNA usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention are oligonucleotides of sufficient length and appropriate sequence so as to provide specific initiation of polymerization on a significant number of nucleic acids in the polymorphic locus. When exposed to appropriate probes or reporters, the sequences that are amplified reveal methylation status and thus diagnostic information. Preferred primers are most preferably eight or more deoxyribonucleotides or ribonucleotides capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The priming segment of the primer or priming sequence is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on factors such as temperature, buffer, cations, and nucleotide composition. The oligonucleotide primers most preferably contain about 12-20 nucleotides although they may contain more or fewer nucleotides, preferably according to well known design guidelines or rules. Primers are designed to be substantially complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequence(s) to hybridize and permit amplification of the genomic locus. The primers are employed in the amplification process. That is, reactions (preferably, an enzymatic chain reaction) that produce greater quantities of target locus relative to the number of reaction steps involved. In a most preferred embodiment, the reaction produces exponentially greater quantities of the target locus. Reactions such as these include the PCR reaction. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. The product of the chain reaction is a discrete nucleic acid duplex with termini corresponding to the ends of the specific primers employed.

The primers may be prepared using any suitable method, such as conventional phosphotriester and phosphodiester methods including automated methods. In one such automated embodiment, diethylphosphoramidites are used as starting materials and may be synthesized as described by Beaucage et al. (1981). A method for synthesizing oligonucleotides on a modified solid support is described in U.S. Pat. No. 4,458,066.

Any nucleic acid specimen taken from urine or urethral wash, in purified or non-purified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA. The DNA or RNA may be single stranded or double stranded. In the event that RNA is to be used as a template, enzymes, and/or conditions optimal for reverse transcribing the template to DNA would be utilized. In addition, a DNA-RNA hybrid containing one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule so that the specific sequence constitutes the entire nucleic acid.

If the extracted sample is impure, it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical or enzymatic means. One physical method of separating nucleic acid strands involves heating the nucleic acid until it is denatured. Typical heat denaturation may involve temperatures ranging from about 80 to 105° C. for up to 10 minutes. Strand separation may also be induced by an enzyme from the class of enzymes known as helicases or by the enzyme RecA, which has helicase activity, and in the presence of riboATP, is known to denature DNA. Reaction conditions that are suitable for strand separation of nucleic acids using helicases are described by Kuhn Hoffmann-Berling (1978). Techniques for using RecA are reviewed in C. Radding (1982). Refinements of these techniques are now also well known.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. A molar excess (for genomic nucleic acid, usually about 10₈:1, primer:template) of the two oligonucleotide primers is preferably added to the buffer containing the separated template strands. The amount of complementary strand may not be known if the process of the invention is used for diagnostic applications, so the amount of primer relative to the amount of complementary strand cannot always be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90-100° C. for up to 10 minutes, preferably from 1 to 4 minutes. After this heating period, the solution is allowed to cool to room temperature, which is preferable for the primer hybridization. To the cooled mixture is added an appropriate agent for effecting the primer extension reaction (the “agent for polymerization”), and the reaction is allowed to occur under conditions known in the art. The agent for polymerization may also be added together with the other reagents if it is heat stable. This synthesis (or amplification) reaction may occur at room temperature up to a temperature at which the agent for polymerization no longer functions. The agent for polymerization may be any compound or system that will function to accomplish the synthesis of primer extension products, preferably enzymes. Suitable enzymes for this purpose include, for example, E. coli DNA polymerase 1, Klenow fragment of E. coli DNA polymerase I, T4 DNA polymerase, other available DNA polymerases, polymerase mutants, reverse transcriptase, and other enzymes, including heat-stable enzymes (e.g., those enzymes which perform primer extension after being subjected to temperatures sufficiently elevated to cause denaturation). A preferred agent is Taq polymerase. Suitable enzymes will facilitate combination of the nucleotides in the proper manner to form the primer extension products complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Most preferably, the method of amplifying is by PCR. Alternative methods of amplification can also be employed as long as the methylated and non-methylated loci amplified by PCR using the primers of the invention is similarly amplified by the alternative means. In one such most preferred embodiment, the assay is conducted as a nested PCR. In nested PCR methods, two or more staged polymerase chain reactions are undertaken. In a first-stage polymerase chain reaction, a pair of outer oligonucleotide primers, consisting of an upper and a lower primer that flank a particular first target nucleotide sequence in the 5′ and 3′ position, respectively, are used to amplify that first sequence. In subsequent stages, a second set of inner or nested oligonucleotide primers, also consisting of an upper and a lower primer, are used to amplify a smaller second target nucleotide sequence that is contained within the first target nucleotide sequence.

The upper and lower inner primers flank the second target nucleotide sequence in the 5′ and 3′ positions, respectively. Flanking primers are complementary to segments on the 3′-end portions of the double-stranded target nucleotide sequence that is amplified during the PCR process. The first nucleotide sequence within the region of the gene targeted for amplification in the first-stage polymerase chain reaction is flanked by an upper primer in the 5′ upstream position and a lower primer in the 3′ downstream position. The first targeted nucleotide sequence, and hence the amplification product of the first-stage polymerase chain reaction, has a predicted base-pair length, which is determined by the base-pair distance between the 5′ upstream and 3′ downstream hybridization positions of the upper and lower primers, respectively, of the outer primer pair.

At the end of the first-stage polymerase chain reaction, an aliquot of the resulting mixture is carried over into a second-stage polymerase chain reaction. This is preferably conducted within a sealed or closed vessel automatically such as with the “SMART CAP” device from Cepheid. In this second-stage reaction, the products of the first-stage reaction are combined with specific inner or nested primers. These inner primers are derived from nucleotide sequences within the first targeted nucleotide sequence and flank a second, smaller targeted nucleotide sequence contained within the first targeted nucleotide sequence. This mixture is subjected to initial denaturation, annealing, and extension steps, followed by thermocycling as before to allow for repeated denaturation, annealing, and extension or replication of the second targeted nucleotide sequence. This second targeted nucleotide sequence is flanked by an upper primer in the 5′ upstream position and a lower primer in the 3′ downstream position. The second targeted nucleotide sequence, and hence the amplification product of the second-stage PCR, also has a predicted base-pair length, which is determined by the base-pair distance between the 5′ upstream and 3′ downstream hybridization positions of the upper and lower primers, respectively, of the inner primer pair.

The amplified products are preferably identified as methylated or non-methylated with a probe or reporter specific to the product as described in U.S. Pat. No. 4,683,195. Advances in the field of probes and reporters for detecting polynucleotides are well known to those skilled in the art.

Optionally, the methylation pattern of the nucleic acid can be confirmed by other techniques such as restriction enzyme digestion and Southern blot analysis. Examples of methylation sensitive restriction endonucleases which can be used to detect 5′ CpG methylation include SmaI, SacII, EagI, MspI, HpaII, BstUI and BssHII.

In another aspect of the invention a methylation ratio is used. This can be done by establishing a ratio between the amount of amplified methylated species of Marker attained and the amount of amplified reference Marker or non-methylated Marker region amplified. This is best done using quantitative real-time PCR. Ratios above an established or predetermined cutoff or threshold are considered hypermethylated and indicative of having a proliferative disorder such as cancer (prostate cancer in the case of GSTP1). Cutoffs are established according to known methods in which such methods are used for at least two sets of samples: those with known diseased conditions and those with known normal conditions. The reference Markers of the invention can also be used as internal controls. The reference Marker is preferably a gene that is constitutively expressed in the cells of the samples such as Beta Actin.

Established or predetermined values (cutoff or threshold values) are also established and used in methods according to the invention in which a ratio is not used. In this case, the cutoff value is established with respect to the amount or degree of methylation relative to some baseline value such as the amount or degree of methylation in normal samples or in samples in which the cancer is clinically insignificant (is known not to progress to clinically relevant states or is not aggressive). These cutoffs are established according to well-known methods as in the case of their use in methods based on a methylation ratio.

Since a decreased level of transcription of the gene associated with the Marker is often the result of hypermethylation of the polynucleotide sequence and/or particular elements of the expression control sequences (e.g., the promoter sequence), primers prepared to match those sequences were prepared. Accordingly, the invention provides methods of detecting or diagnosing a cell proliferative disorder by detecting methylation of particular areas, preferably, within the expression control or promoter region of the Markers. Probes useful for detecting methylation of these areas are useful in such diagnostic or prognostic methods.

The kits of the invention can be configured with a variety of components provided that they all contain at least one primer or probe or a detection molecule (e.g., Scorpion reporter). In one embodiment, the kit includes reagents for amplifying and detecting hypermethylated Marker segments. Optionally, the kit includes sample preparation reagents and/or articles (e.g., tubes) to extract nucleic acids from samples.

In a preferred kit, reagents necessary for one-tube MSP are included such as, a corresponding PCR primer set, a thermostable DNA polymerase, such as Taq polymerase, and a suitable detection reagent(s) such as hydrolysis probe or molecular beacon. In optionally preferred kits, detection reagents are Scorpion reporters or reagents. A single dye primer or a fluorescent dye specific to double-stranded DNA such as ethidium bromide can also be used. The primers are preferably in quantities that yield high concentrations. Additional materials in the kit may include: suitable reaction tubes or vials, a barrier composition, typically a wax bead, optionally including magnesium; necessary buffers and reagents such as dNTPs; control nucleic acid(s) and/or any additional buffers, compounds, co-factors, ionic constituents, proteins and enzymes, polymers, and the like that may be used in MSP reactions. Optionally, the kits include nucleic acid extraction reagents and materials.

A Biomarker is any indicia of an indicated Marker nucleic acid/protein. Nucleic acids can be any known in the art including, without limitation, nuclear, mitochondrial (homeoplasmy, heteroplasmy), viral, bacterial, fungal, mycoplasmal, etc. The indicia can be direct or indirect and measure over- or under-expression of the gene given the physiologic parameters and in comparison to an internal control, placebo, normal tissue or another carcinoma. Biomarkers include, without limitation, nucleic acids and proteins (both over and under-expression and direct and indirect). Using nucleic acids as Biomarkers can include any method known in the art including, without limitation, measuring DNA amplification, deletion, insertion, duplication, RNA, microRNA (miRNA), loss of heterozygosity (LOH), single nucleotide polymorphisms (SNPs, Brookes (1999)), copy number polymorphisms (CNPs) either directly or upon genome amplification, microsatellite DNA, epigenetic changes such as DNA hypo- or hyper-methylation and FISH. Using proteins as Biomarkers includes any method known in the art including, without limitation, measuring amount, activity, modifications such as glycosylation, phosphorylation, ADP-ribosylation, ubiquitination, etc., or immunohistochemistry (IHC) and turnover. Other Biomarkers include imaging, molecular profiling, cell count and apoptosis Markers.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with an indication or tissue type.

Preferred methods for establishing gene expression profiles include determining the amount of RNA that is produced by a gene that can code for a protein or peptide. This is accomplished by reverse transcriptase PCR(RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All Markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio of less than one (down-regulation) appears in the blue portion of the spectrum while a ratio of greater than one (up-regulation) appears in the red portion of the spectrum. Commercially available computer software programs are available to display such data including “Genespring” (Silicon Genetics, Inc.) and “Discovery” and “Infer” (Partek, Inc.)

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the algorithm. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Methods of isolating nucleic acid and protein are well known in the art. See e.g. the discussion of RNA found at the Ambion website on the Worldwide Web and in US and 20070054287.

DNA analysis can be any known in the art including, without limitation, methylation, de-methylation, karyotyping, ploidy (aneuploidy, polyploidy), DNA integrity (assessed through gels or spectrophotometry), translocations, mutations, gene fusions, activation-de-activation, single nucleotide polymorphisms (SNPs), copy number or whole genome amplification to detect genetic makeup. RNA analysis includes any known in the art including, without limitation, q-RT-PCR, miRNA or post-transcription modifications. Protein analysis includes any known in the art including, without limitation, antibody detection, post-translation modifications or turnover. The proteins can be cell surface markers, preferably epithelial, endothelial, viral or cell type. The Biomarker can be related to viral/bacterial infection, insult or antigen expression.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The GSTP1 assays of the instant application showed greatly improved assay performance in the samples tested. A combination of more than one design for the same gene (GSTP1) shows improved clinical sensitivity with a very high specificity. A combination of two assays for the same gene provides a less complex solution to achieving a better clinical sensitivity with fewer genes targeted in a given multiplex with a very high specificity. Better assay performance with multiple assay designs for GSTP1 provides the ability to remove other marker genes from the multiplex leading to a higher specificity. Improved clinical sensitivity at a high specificity will yield better negative and positive predictive values.

The following examples are provided to illustrate but not limit the invention.

### Example 1

Two new designs (Version 2 and Version 3) were compared to the existing design (Version 1) in FFPE tissue samples. All three designs are shown in Table 1 below.

TABLE 1
Description of GSTP1 assay designs

| | | | |
|---|---|---|---|
| Sequence Name | Scorpion/Primer | Name | Seq ID No |

| | | | |
|---|---|---|---|
| GSTP1_Fam_Sc_AS_1112 | FAM-CGCACGGCGAACTCCCGCCGACGTGCG BHQ-HEG- | Version 1 | 1 |
| | TGTAGCGGTCGTCGGGGTTG | | |
| GSTP1_1151_L22 | 5′ GCCCCAATACTAAATCACGACG 3′ | | 2 |
| GSTP1_Sc_M_S_1207 | FAM-CCGGTCGCGAGGTTTTCGACCGG-BHQ-HEG- | Version 2 | 3 |
| | CCGAAAAACGAACCGCGCGTA | | |
| GSTP1_1179_U27 | GGGCGGGATTATTTTTATAAGGTTCGG | | 4 |
| GSTP1_Sc_M_AS_888 | FAM-CGGCCCTAAAACCGCTACGAGGGCCG-BHQ-HEG- | Version 3 | 5 |
| | GAAGCGGGTGTGTAAGTTTCGG | | |
| GST_929_L26 | ACGAAATATACGCAACGAACTAACGC | | 6 |
| APC_M_781_AS15_TR | Texas Red - GCCGGCGGGTTTTCGACGGGCCGGC-BHQ2-HEG- | | 7 |
| | CGAACCAAAACGCTCCCCA | | |
| APC_804_L25 | GTCGGTTACGTGCGTTTATATTTAG | | 8 |
| Actin_Q670_Sc_382_L15 | Q670-CCGCGCATCACCACCCCACACGCGCGG-BHQ2-HEG- | | 9 |
| | GGAGTATATAGGTTGGGGAAGTTTG | | |
| Actin_425_L27 | 5′ AACACACAATAACAAACACAAATTCAC 3′ | | 10 |

Experiments were run with Version 1 design in Fam in a triplex assay with APC and Actin and each of the new GSTP1 designs (Version 2 and Version 3) in singlex in the Fam channel on the Cepheid SmartCycler® system. 33 adenocarcinomas from radical prostatectomies and 20 negative biopsies were tested. Taq DNA Polymerase conjugated to TP6-25 antibody as a hot-start mechanism was used. Resulting data from this set of samples shows that the two newer assay designs have improved sensitivity compared to the original Version 1 design. Data is shown below in Table 2 as a summary.

TABLE 2
Summary of data with Version 1 design in a triplexed assay and
Version 2 and 3 designs in singlex on the Cepheid platform

| | GSTP1 | | |
|---|---|---|---|
| | Version 3 GSTP1 | Version 2 GSTP1 | Version 1 GSTP1 |
| Sensitivity | 90.91% | 90.91% | 63.64% |
| Specificity | 100.00% | 100.00% | 100.00% |
| | 3 Gene Combo | 3 Gene Combo | |
| | V3 GSTP1 + V1 | V2 GSTP1 + V1 | 3 Gene Combo |
| | GSTP1 + APC | GSTP1 + APC | V1 GSTP1/APC |
| Sensitivity | 90.91% | 90.91% | 84.85% |
| Specificity | 100.00% | 100.00% | 100.00% |

Further optimization of the assay showed that switching to FastStart Taq enzyme improved the clinical sensitivity of GSTP1 in the assay. All reactions were set up using these optimized conditions for all experiments going forward. These reaction conditions are shown below.

### Assay Master Mix Buffer Formulation

| | Stock | | Final | |
|---|---|---|---|---|
| Component | Concentration | | Concentration | |
| Nuclease Free Water | | | | |
| D (+) Trehalose | 1.5 | Molar | 150 | mM |
| Tris-HCl, pH 8 | 1 | Molar | 46.8 | mM |
| Magnesium Chloride Solution | 1 | Molar | 3.5 | mM |
| Tween-20 | 10% | | 0.2% | |
| dNTP mix | 25 | mM each | 123 | uM |
| ProClin 300 | 10% | | 0.06% | |
| DMSO | 100% | | 5% | |

### Assay Enzyme Mix Formulation

| | Stock | | Final | |
|---|---|---|---|---|
| Component | Concentration | | Concentration | |
| Nuclease Free Water | | | | |
| Tris-HCl, pH 8 | 1 | Molar | 16 | mM |
| BSA | 10% | | 0.05% | |
| KCL | 2 | Molar | 10 | mM |
| FastStart Taq Polymerase | 5 | U/ul | 1 | U/ul |
| ProClin 300 | 10% | | 0.008% | |

### Assay Primer/Probe Formulation

| | Stock Conc. | P/P Mix Final | |
|---|---|---|---|
| Component | (MW) | Conc. | Example |
| GSTP1-1 Primer | 100 uM | 10 uM | 10 ul |
| GSTP1-1 Scorpion | 100 uM | 10 uM | 10 ul |
| GSTP1-3 Primer | 100 uM | 10 uM | 10 ul |
| GSTP1-3 Scorpion | 100 uM | 10 uM | 10 ul |
| BACTIN Primer | 100 uM | 10 uM | 10 ul |
| BACTIN Scorpion | 100 uM | 10 uM | 10 ul |
| Tris pH 8 | 10 mM | | 40 ul |
| | | Total = | 100 ul |

### Reaction Mix:

Reaction Mix

| Reagents | 1 rxn (ul) |
|---|---|
| FS PCR Buffer | 10 |
| FS enzyme mix | 5 |
| Primer/Probe Mix | 1.25 |
| Water | 3.75 |
| Total | 20 |

### Cycling Conditions:

Cycling Conditions

95 C 240 s
40 cycles
95 C 15 s
61 C 30 s

Clinical samples were run with FastStart Taq on the Cepheid platform with bisulfite modified DNA from 67 adenocarcinomas obtained from radical prostatectomies, 36 normal tissues from radical prostatectomies, and 24 negative prostate biopsies. Two assays were run on these samples with one assay being a multiplex with Version 2 GSTP1 (Fam), Version 3 GSTP1 (Texas Red), and Actin (Q670) and the second multiplexed assay with a combination of Version 1 GSTP1 (Fam), APC (Q570), and Actin (Q670). Resulting data is summarized in Table 3.

TABLE 3
Performance of each of the GSTP1 designs
and APC in a multiplexed assay

| | V2 GSTP1 | V3 GSTP1 | V1 GSTP1 | APC |
|---|---|---|---|---|
| Sensitivity | 85% | 78% | 76% | 78% |
| Specificity | 98% | 97% | 100% | 97% |

When the same set of data was analyzed to determine whether multiple GSTP1 designs contribute to better clinical sensitivity, better assay performance was indeed observed and the data is summarized in Table 4.

TABLE 4
Performance of multiple GSTP1 designs in a multiplexed assay

| | V2 GSTP1 + V3 | V2 | V3 | V1 | V2GSTP1 + V3 | V1 GSTP1 + V3 |
|---|---|---|---|---|---|---|
| | GSTP1 | GSTP1 + APC | GSTP1 + APC | GSTP1 + APC | GSTP1 + APC | GSTP1 + APC |
| Sensitivity | 90% | 91% | 93% | 90% | 94% | 93% |
| Specificity | 97% | 97% | 97% | 97% | 97% | 97% |

In order to have a better comparison between the two new GSTP1 designs and determine whether or not APC adds value to the multiplex when the two new GSTP1 designs are used, an experiment was run with the same sample set with a multiplex that included GSTP1 Version2 (Fam), GSTP1 Version 3 (Cy3), APC, and Actin. A total of 38 adenocarcinomas and 36 normal samples obtained from radical prostatectomies were tested. Data is summarized in Table 5.

TABLE 5
Assay performance of two GSTP1 designs vs. two GSTP1 designs with APC

| | | | | V2GSTP1 + V3 | V2GSTP1 + V3 |
|---|---|---|---|---|---|
| | V2 GSTP1 | V3 GSTP1 | APC | GSTP1 + APC | GSTP1 |
| Sensitivity | 74% | 68% | 61% | 89% | 87% |
| Specificity | 97% | 94% | 97% | 94% | 94% |

Data shown above demonstrate the complementary performance of the two GSTP1 designs to each other. The combination of the two GSTP1 designs delivers a performance very close to having a two gene combination such as GSTP1 and APC together. This is a novel application whereby more than one assay can target the same gene with high specificity and yield improved clinical sensitivity. This leads to an application where a different complementing marker is not needed to achieve high sensitivity at a very high specificity. GSTP1 hypermethylation is known to be very specific to cancer in prostate tissues whereas APC could lead to lower specificity. Therefore an assay with just GSTP1 and a housekeeping gene is likely to provide a comparable clinical sensitivity at a higher specificity, than, for example, a combination of GSTP1 with APC in initial negative biopsies that are subsequently positive. When negative biopsies are tested with this assay high specificity becomes extremely important.

### REFERENCES CITED

20020197639
20030022215
20030032026
20030082600
20030087258
20030096289
20030129620
20030148290
20030157510
20030170684
20030194734
20030215842
20030224040
20030232351
20040023279
20040038245
20040048275
20040072197
20040086944
20040101843
20040115663
20040132048
20040137474
20040146866
20040146868
20040152080
20040171118
20040203048
20040241704
2004248090
20040248120
20040265814
20050009059
20050019762
20050026183
20050053937
20050064428
20050069879
20050079527
20050089870
20050130172
20050153296
20050196792
20050208491
20050208538
20050214812
20050233340
20050239101
20050260630
20050266458
20050287553
20070054287
U.S. Pat. No. 4,458,066
U.S. Pat. No. 4,683,195
U.S. Pat. No. 5,242,974
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,436,327
U.S. Pat. No. 5,445,934
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,556,752
U.S. Pat. No. 5,561,071
U.S. Pat. No. 5,571,639
U.S. Pat. No. 5,593,839
U.S. Pat. No. 5,599,695
U.S. Pat. No. 5,624,711
U.S. Pat. No. 5,658,734
U.S. Pat. No. 5,700,637
U.S. Pat. No. 5,786,146
U.S. Pat. No. 6,004,755
U.S. Pat. No. 6,136,182
U.S. Pat. No. 6,214,556
U.S. Pat. No. 6,218,114
U.S. Pat. No. 6,215,122
U.S. Pat. No. 6,251,294
U.S. Pat. No. 6,271,002
U.S. Pat. No. 6,331,393
U.S. Pat. No. 6,335,165
U.S. Pat. No. 6,992,182
Bastian, P et al. (2004) Molecular biomarker in prostate cancer: the role of CpG island hypermethylation Eur Urol 46:698-708
Beaucage et al. (1981) Tetrahedron Letters 22:1859-1862
Brookes (1999) The essence of SNPs Gene 234:177-186
Chan, Q et al. (2005) Promoter methylation and differential expression of π-class glutathione S-transferase in endometrial carcinoma J Mol Diagn 7:8-16
Coles et al. (1990) The role of glutathione and glutathione transferases in chemical carcinogenesis CRC Crit. Rev Biochem Mol Biol 25:47
Das, R et al. (2006) Computational prediction of methylation status in human genomic sequences PNAS103:10713-10716
Eckhardt, F et al. (2006) DNA methylation profiling of human chromosomes 6, 20, and 22 Nature Genetics 38:1378-1385
Endoh, M. et al. (2005) RASSF2, a potential tumor suppressor, is silenced by CpG island hypermethylation in gastric cancer Br J Cancer 93:1395-1399
Esteller, M (2005) Aberrant DNA methylation as a cancer-inducing mechanism Ann Rev Pharmacol Toxicol 45:629-656
Esteller, M et al. (2001) A gene hypermethylation profile of human cancer Cancer Res 61:3225-3229
Guo, M et al. (2006) Accumulation of promoter methylation suggests epigenetic progression in squamous cell carcinoma of the esophagus Clin Can Res 12:4515-4522
Harden, S et al. (2003) Quantitative GSTP1 Methylation and the Detection of Prostate Adenocarcinoma in Sextant Biopsies J Natl Cancer Inst 95:1634-1637
Herman et al. (1996) Methylation-specific PCR: a novel PCR assay for methylation status of CpG islands Proc Natl Acad Sci USA 93:9821
Jerònimo, C et al. (2001) Quantitation of GSTP1 Methylation in Non-neoplastic Prostatic Tissue and Organ-Confined Prostate Adenocarcinoma J Natl Cancer Inst 93:1747-1752
Jeronimo, C et al. (2004) Quantitative RARbeta2 hypermethylation: a promising prostate cancer marker Clin Cancer Res 10:4010-4014
Jones et al. (2002) The fundamental role of epigenetic events in cancer Nature Rev 3:415.
Kuhn Hoffmann-Berling (1978) CSH-Quantitative Biology 43:63
Lee, W et al. (1994) Cytidine methylation of regulatory sequences near the pi-class glutathione S-transferase gene accompanies human prostatic carcinogenesis Proc Natl Acad Sci USA
Mannervik et al. (1992) Nomenclature for human glutathione transferases Biochem J 282:305
Markowitz (1952) Portfolio Selection
Nakayama, M et al. (2003) Hypermethylation of the human glutathione S-transferase-π gene (GSTP1) CpG island is present in a subset of proliferative inflammatory atrophy lesions but not in normal or hyperplastic epithelium of the prostate Am J Pathol 163:923-933
Nakayama, T et al. (2001) The role of epigenetic modifications in retinoic acid receptor beta2 gene expression in human prostate cancers Lab Invest 81:1049-1057
Pao, M et al. (2001) The endothelin receptor B (EDNRB) promoter displays heterogeneous, site specific methylation patterns in normal and tumor cells Hum Molec Genet 10:903-910
Pickett et al. (1989) Glutathione S-transferases: gene structure, regulation, and biological function Annu Rev Biochem 58:743
Radding (1982) Homologous pairing and strand exchange in genetic recombination Ann Rev Genet 16:405-437
Rushmore et al. (1993) Glutathione S-transferases, structure, regulation, and therapeutic implications J Biol Chem 268:11475
Satoh et al. (2002) DNA methylation and histone deacetylation associated with silencing DAP kinase gene expression in colorectal and gastric cancers Brit J Cancer 86:1817-1823
Shaw, R et al. (2006) Promoter methylation of p 16, RARβ, E-cadherin, cyclin A1 and cytoglobin in oral cancer: quantitative evaluation using pyrosequencing Br J Can 94:561-568
Strunnikova, M et al. (2005) Chromatin inactivation precedes de novo DNA methylation during the progressive epigenetic silencing of the RASSF1A promoter Molec Cellular Biol 25:3923-33933
Warnecke et al. (1997) Detection and measurement of PCR bias in quantitative methylation analysis of bisulphite-treated DNA Nucl Acids Res 25:4422-4426 www.ambion.come/techlib/basics/rnaisol/index.html
Yan, P et al. (2003) Differential distribution of DNA methylation within the RASSF1A CpG island in breast cancer Can Res 63:6178-6186
Yu, J et al. (2006) DNA repair pathway profiling and microsatellite instability in colorectal cancer Clin Can Res 12:5104-5111

We claim:

1. A prostate cancer assay having reagents for the detection of multiple methylation sites wherein each target is directed to a different part of the same gene.

2. The assay of claim 1 wherein the gene is GSTP1.

3. The assay of claim 2 wherein at least one methylation site is in the promoter region.

4. The assay of claim 2 wherein two or more methylation sites are in the promotion region.

5. The assay of claim 1 wherein reagents detect at least three different methylation sites.

6. The assay of claim 1 having reagents for the detection of only multiple methylation sites directed to a different part of the same gene.

7. The assay of claim 6 wherein the gene is GSTP1.

8. The assay of claim 7 wherein at least one methylation site is in the promoter region.

9. The assay of claim 7 wherein two or more methylation sites are in the promotion region.

10. The assay of claim 7 wherein reagents detect at least three different methylation sites.

11. A prostate cancer assay having reagents for the detection of multiple methylation sites comprising Seq. ID No. 2 and Seq. ID No. 4.

12. The assay of claim 12 further comprising reagents for the detection of multiple methylation sites comprising Seq. ID No.6.

13. A prostate cancer assay having reagents for the detection of multiple methylation sites comprising Seq. ID No. 2 and Seq. ID No. 6.

14. The assay of claim 13 further comprising reagents for the detection of multiple methylation sites comprising Seq. ID No.4.

15. A kit comprising reagents for detecting multiple methylated nucleotide sequences from within the same gene and conversion reagents.

16. The kit of claim 15 wherein the conversion reagents include sodium bisulfite.

17. The kit of claim 16 wherein the gene is GSTP1.

18. The kit of claim 16 wherein said reagents are designed for use with FFPE tissue samples.

19. The kit of claim 18 wherein the samples are prostate samples.

20. The kit of claim 16 wherein said reagents are designed for use with urine samples.


SEQUENCE LISTING (10 sequences)

SEQ ID NO: 1 - DNA, 47 bp, human
cgcacggcgaactcccgccgacgtgcgtgtagcggtcgtcggggttg

SEQ ID NO: 2 - DNA, 22 bp, human
gccccaatactaaatcacgacg

SEQ ID NO: 3 - DNA, 44 bp, human
ccggtcgcgaggttttcgaccggccgaaaaacgaaccgcgcgta

SEQ ID NO: 4 - DNA, 27 bp, human
gggcgggattatttttataaggttcgg

SEQ ID NO: 5 - DNA, 48 bp, human
cggccctaaaaccgctacgagggccggaagcgggtgtgtaagtttcgg

SEQ ID NO: 6 - DNA, 26 bp, human
acgaaatatacgcaacgaactaacgc

SEQ ID NO: 7 - DNA, 44 bp, human
gccggcgggttttcgacgggccggccgaaccaaaacgctcccca

SEQ ID NO: 8 - DNA, 25 bp, human
gtcggttacgtgcgtttatatttag

SEQ ID NO: 9 - DNA, 52 bp, human
ccgcgcatcaccaccccacacgcgcggggagtatataggttggggaagtttg

SEQ ID NO: 10 - DNA, 27 bp, human
aacacacaataacaaacacaaattcac